(19) United States
(12) Patent Application Publication
(10) Pub. No.: US 2008/0296145 A1
(43) Pub. Date: Dec. 4, 2008

(54) Method for Improving the Color Number

(75) Inventors: Rolf Pinkos, Bad Durkheim (DE); Harald Rust, Neustadt (DE); Gerd-Dieter Tebben, Mannheim (DE); Alexander Hauk, Ludwigshafen (DE); Christian Muller, Mannheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/089,304

(22) PCT Filed: Oct. 6, 2006
(86) PCT No.: PCT/EP06/67153

§ 371 Date: Apr. 4, 2008

(30) Foreign Application Priority Data
Oct. 7, 2005 (DE) 10 2005 048 250.3

(51) Int. Cl. C07C 49/413

(52) U.S. Cl. 204/158.21

(57) ABSTRACT

The present invention relates to a process for purifying a composition (I) at least comprising cyclododecanone. The process according to the invention for purifying a composition (I) at least comprising cyclododecanone comprises at least step (i)
(i) irradiation of the composition (I).

The present invention relates to a process for purifying a composition (I) at least comprising cyclododecanone.

Cyclododecanone is required in high purity for various applications. For example, cyclododecanone is an important intermediate for the preparation of, for example, laurolactam, dodecanedicarboxylic acid and polyamides derived therefrom, for example nylon-12 or nylon-6,12.

Cyclododecanone is prepared, for example, by air oxidation of cyclododecane in the presence of boric acid to give cyclododecyl borate, hydrolysis of the borate to give cyclododecanol and subsequent dehydrogenation of the cyclododecanol. Cyclododecane itself is also obtained by full hydrogenation of cyclodecatriene. One description of this industrial process for synthesizing cyclododecanone can be found in T. Schiffer, G. Oenbrink, “cyclododecanol, cyclododecanone and laurolactam” in Ullmann's Encyclopedia of Industrial Chemistry, 6th Edition, 2000, Electronic Release, Wiley VCH.

A further process starts from the epoxidation of cyclododecatriene, in which cyclododecanone is obtained from the epoxide after hydrogenation and rearrangement. Such a process is disclosed, for example, in EP 1 018 498 A2.

DE 103 44 595 A and DE 103 44 594 A describe processes for preparing cyclododecanone, in which an oxidation with dinitrogen monoxide is effected in one process step.

A typical quality feature of chemical products, which also relates to the purity of the products, is the so-called color number which is characterized, for example, by the so-called APHA number according to DIN standard 53409.

In many products, a minimum color number is required, which often requires laborious additional purifying operations. For example, the so-called color number hydrogenation is widespread, but this means a considerable level of complexity, so that the process is costly and inconvenient.

Undesired high color numbers are often obtained even in purifying distillation, for example as a result of thermal stress and/or as a result of action of air through not entirely leak-free columns.

EP 0 285 372 A2 discloses a process in which cyclic ketones are purified by irradiation with light. The examples mention only cyclopentadecanone.

It was an object of the present invention to provide a process with which cyclododecanone can be obtained in high purity in a simple manner and with a low level of complexity, and has a sufficiently low color number.

It was a further object of the present invention to provide a purifying process for cyclododecanone which can be combined in a simple manner with known preparation processes or purification processes for cyclododecanone.

This object is achieved in accordance with the invention by a process for purifying a composition (I) at least comprising cyclododecanone, at least comprising step (i)
(i) irradiation of the composition (I).

It has been found that the color number of a composition comprising cyclododecanone can be improved in a simple manner by exposing the product to irradiation.

The wavelength in the irradiation can be varied within wide ranges. The wavelength of the light used should advantageously be selected such that the light absorption range of the cyclododecanone corresponds with the wavelength of the incident light. According to the invention, for example, irradiation is effected with light of a wavelength of from 200 to 800 nm, preferably from 250 to 550 nm, in particular from 300 to 470 nm, more preferably from 400 to 460 nm.

In a further embodiment, the invention therefore further relates to a process as described above for purifying a composition (I) at least comprising cyclodecanone, wherein the irradiation is effected with light of a wavelength of from 200 to 800 nm.

According to the invention, the light source used may be any suitable radiation source, especially a radiation source which radiates light of a wavelength between 200 and 800 nm. In principle, useful radiation sources in the context of the present invention are, for example, sunlight, light from incandescent lamps or neon tubes or light from mercury lamps. Suitable radiation sources are also UV lamps, xenon lamps, fluorescent lamps, indium lamps or metal halide lamps.

According to the invention, the duration of the irradiation is determined according to the original color number of the composition (I) before the irradiation, according to the desired color number or according to the wavelength of the incident light. According to the invention, the irradiation in step (i) is effected for at least 1 minute. A suitable irradiation step is effected, for example, for from 0.1 to 48 hours, preferably from 0.5 to 30 hours, in particular from 1 to 24 hours, more preferably from 1.5 to 12 hours.

In a preferred embodiment of the present invention, the irradiation in step (i) is effected with light of a wavelength of from 300 to 470 nm and for from 0.1 to 20 hours, preferably from 0.1 to 15 hours, in particular from 0.1 to 10 hours, more preferably from 0.1 to 5 hours. In a further preferred embodiment of the present invention, the irradiation in step (i) is effected with light of a wavelength of from 400 to 460 nm and for from 0.1 to 10 hours, preferably from 0.1 to 5 hours, in particular from 0.1 to 2 hours, more preferably from 0.1 to 1 hour.

In a further embodiment, the present invention therefore relates to a process as described above for purifying a composition (I) at least comprising cyclododecanone, wherein the irradiation is effected for from 0.1 to 48 hours.

According to the invention, the composition (I) may comprise further components. These components may be those which are not attacked in the inventive purification process and those which are removed in the inventive purification process. The composition (I) may comprise, for example, organic compounds, especially those having oxygen-containing functional groups. For example, the composition (I) may comprise epoxides, aldehydes, alcohols or diketones, especially cyclic diketones. The organic compounds may in particular have the same number of carbon atoms as the cyclododecanone present in the composition (I).

In a further embodiment, the present invention therefore relates to a process as described above for purifying a composition (I) at least comprising cyclododecanone, wherein the composition (I) comprises at least one cyclic diketone.

In this case, the composition (I) comprises cyclododecanone typically in an amount of more than 80% by weight, preferably from 85 to 99.9% by weight, in particular from 90 to 99.5% by weight, more preferably from 92 to 99% by weight.

Before the inventive purification is carried out, the secondary components are present in the composition (I) in particular to an extent of less than 20% by weight, in particular less than 15% by weight, more preferably less than 10% by weight. For example, the secondary components are present in an amount of from 0.001 to 9% by weight, preferably of from 0.01 to 5% by weight.

The inventive purification process improves the color number of the composition (I). After the irradiation in step (i), the composition (i) preferably has a color number of less than 50 APHA, preferably of from 0 to 40 APHA, in particular of from 1 to 15 APHA, preferably of from 1.5 to 12 APHA, more preferably of from 2 to 10 APHA.

In a further embodiment, the present invention therefore relates to a process as described above for purifying a composition (I) at least comprising cyclododecanone, wherein the composition (I), after the irradiation according to step (i), has a color number of from 0 to 40 APHA.

The process according to the invention may be carried out batchwise or else continuously. It is advantageous for the products to be treated to be liquid, but this is not absolutely necessary.

The composition (I) may be dissolved in a solvent and then subjected to the irradiation in (i).

The process according to the invention may in principle be performed at any temperature and any pressure. The irradiation in (i) is preferably effected at standard pressure. The irradiation in (i) can be effected, for example, at a temperature of below 60° C., in which case the product is preferably solid. Preference is given to effecting the irradiation in (i) at a temperature of above 60° C., in which case the composition (I) is preferably liquid. The temperature in the irradiation in (i) is more preferably between 62 and 150° C., in particular between 65 and 130° C.

According to the invention, the process may also comprise at least one further distillation or crystallization or a distillation and a crystallization of the composition (I).

In a further embodiment, the present invention therefore relates to a process as described above for purifying a composition (I) at least comprising cyclododecanone, which comprises at least one step (ii):
(ii) distillation and/or crystallization of the composition (I).

According to the invention, step (ii) may be performed before or after step (i). It is also possible that a distillation or crystallization as per (ii) is effected before and after step (i).

The distillation and/or crystallization may be carried out by all customary processes known to those skilled in the art.

Suitable solvents for the crystallization in stage (ii) are, for example, alcohols, ethers, hydrocarbons, aromatic hydrocarbons, ketones. When a solvent is used, preference is given to using toluene, xylene, methanol, ethanol, propanol, butanol, acetone, diethyl ketone or methyl tert-butyl ether. Preference is given to not using any solvent, but instead to carrying out a melt crystallization.

The distillative purification may be effected in one or more columns. Preference is given to working at pressures between 1 and 2000 mbar, for example between 5 and 500 mbar, more preferably between 10 and 200 mbar. The temperatures (bottom temperature) are between 100 and 300° C., preferably between 130 and 250° C., more preferably between 150 and 220° C.

When a column is used, the product of value is preferably obtained via a side draw (liquid or gaseous). High boilers are removed via the bottom, low boilers via the top. When two columns are used, the product of value together with high boilers preferably goes via the bottom into the second column from which it is then obtained via the top or again as a side draw. It is also possible to use dividing wall columns.

By virtue of the process according to the invention, cyclododecanone can be obtained with a purity of, for example, >99.5%. The process according to the invention can be carried out especially after a preparation process or purification process known in principle for cyclododecanone. As a result, the process according to the invention can be combined readily with existing plants, so that no costly modifications are required.

The composition (I) comprising at least cyclododecanone may be obtained by means of all customary preparation processes for cyclododecanone.

For example, cyclododecanone can be obtained by air oxidation of cyclododecane in the presence of boric acid. Cyclododecanone can, for example, also be prepared by hydrogenating cyclododecadienone, by oxidizing cyclododecane or by hydrogenating cyclododecatriene epoxide.

In a further embodiment, the present invention relates to a process as described for purifying a composition (I) at least comprising cyclododecanone, wherein the composition (I) is obtainable by a process at least comprising the stages of
(a-1) trimerization of butadiene to cyclododecatriene
(a-2) oxidation of cyclododecatriene to cyclododecadienone
(a-3) hydrogenation of cyclododecadienone to cyclododecanone.

According to the invention, the composition (I) at least comprising cyclododecanone is obtained preferably by means of a hydrogenation of cyclododecadienone as per stage (a-3) which has in turn been obtained by oxidation of a cyclododecatriene, preferably with dinitrogen monoxide, as per stage (a-2). According to the invention, cyclododecatriene is obtained preferably by trimerizing butadiene as per stage (a-1). According to the invention, further treatments, for example purification steps, may be effected between stages (a-1), (a-2) and (a-3).

Stage (a-1) comprises the trimerization of butadiene. 1,5,9-Cyclododecatriene can be prepared, for example, by trimerizing pure 1,3-butadiene, as described, for example, in T. Schiffer, G. Oenbrink, “Cyclododecatriene, cyclooctadiene and 4-vinylcyclohexene”, Ullmann's Encyclopedia of Industrial Chemistry, 6th Edition (2000), Electronic Release, Wiley VCH. This process forms, in the trimerization in the presence of Ziegler catalysts for example, cis,trans,trans-1,5,9-cyclododecatriene, cis,cis,trans-1,5,9-cyclododecatriene and all-trans-1,5,9-cyclododecatriene, as described, for example, in H. Weber et al. “Zur Bildungsweise von cis,trans,trans-Cyclododecatrien-(1.5.9) mittels titanhaltiger Katalysatoren” in: Liebigs Ann. Chem. 681 (1965) p. 10-20. Cyclododecatriene can be prepared by trimerizing 1,3-butadiene using a titanium catalyst.

While all suitable titanium catalysts can in principle be used for trimerization, the titanium tetrachloride/ethylaluminum sesquichloride catalyst described in the article by Weber et al. is particularly suitable.

The butadiene used for the trimerization especially preferably has a purity determined by gas chromatography of at least 99.6% and more preferably of at least 99.65%. Especially preferably, the 1,3-butadiene used does not comprise any 1,2-butadiene nor any 2-butyne within the precision of detection.

This trimerization generally affords mixtures which comprise at least 95% by weight, preferably at least 96% by weight and more preferably at least 97% by weight of cis,trans,trans-1,5,9-cyclododecatriene. For example, the mixtures especially preferably comprise about 98% by weight of cis,trans,trans-1,5,9-cyclododecatriene.

This mixture comprising cis,trans,trans-1,5,9-cyclododecatriene may be used as such for the reaction in stage (a-2). It is equally possible to remove the cis,trans,trans-1,5,9-cyclododecatriene from the mixture by means of at least one distillation, and to use it in the reaction in stage (a-2).

The oxidation in stage (a-2) can be carried out by any suitable processes known to those skilled in the art. In the process according to the invention, preference is given to carrying out the oxidation in stage (a-2) by means of dinitrogen monoxide.

In stage (a-2), cyclododecatriene is oxidized, preferably by reaction with dinitrogen monoxide. For the reaction of the cyclododecatriene with dinitrogen monoxide, at least one suitable solvent or diluent may be used. These include cyclododecane or cyclododecanone or saturated aliphatic or aromatic, optionally alkyl-substituted hydrocarbons, suitable solvents or diluents being substantially all common solvents and/or diluents with the proviso that they have neither a C—C double bond nor a C—C triple bond nor an aldehyde group.

In general, there is no need to add a solvent or diluent in the reaction of cyclododecatriene with dinitrogen monoxide.

The temperatures in the reaction of cyclododecatriene with dinitrogen monoxide are preferably in the range from 140 to 350° C., more preferably in the range from 180 to 320° C. and particularly preferably in the range from 200 to 300° C.

It is possible to carry out the reaction of cyclododecatriene with dinitrogen monoxide at two or more temperatures or in two or more temperature ranges, each of which is within the above-specified limits. Temperature changes in the course of the reaction can be performed continuously or else discontinuously.

The pressures in the reaction of cyclododecatriene with dinitrogen monoxide are preferably higher than the autogenous pressure of the reactant or product mixture at the selected reaction temperature or the selected reaction temperatures. The pressures are preferably in the range from 1 to 1000 bar, more preferably in the range from 40 to 300 bar and particularly preferably in the range from 50 to 200 bar.

It is possible to carry out the reaction of cyclododecatriene with dinitrogen monoxide at two or more pressures or in two or more pressure ranges, each of which is within the above-specified limits. Pressure changes in the course of the reaction can be performed continuously or else discontinuously.

With regard to the reactors usable for the reaction of cyclododecatriene with dinitrogen monoxide, there are no particular restrictions. In particular, the reaction can be effected in batchwise mode or in continuous mode. Accordingly, the reactors used may, for example, be at least one CSTR (Continuous Stirred Tank Reactor) with at least one internal and/or at least one external heat exchanger, at least one tubular reactor or at least one loop reactor. It is equally possible to configure at least one of these reactors in such a way that it has at least two different zones. Such zones may differ, for example, in reaction conditions, for example the temperature or the pressure and/or in the geometry of the zone, for example the volume or the cross section. When the reaction is carried out in two or more reactors, it is possible to use two or more identical reactor types or at least two different reactor types.

Preference is given to carrying out the reaction of cyclododecatriene with dinitrogen monoxide in a single reactor. For example, preference is given to the reaction in continuous mode.

The residence time of the reactants in the at least one reactor in the reaction of cyclododecatriene with dinitrogen monoxide is generally in the range of up to 20 h, preferably in the range from 0.1 to 20 hours, more preferably in the range from 0.2 to 15 hours and particularly preferably in the range from 0.25 to 10 h.

In the feed which is fed to the reaction of dinitrogen monoxide with cyclododecatriene, the molar ratio of dinitrogen monoxide to cyclododecatriene is generally in the range from 0.05 to 4, preferably in the range from 0.06 to 1, more preferably in the range from 0.07 to 0.5 and particularly preferably in the range from 0.1 to 0.4.

The reaction of cyclododecatriene with dinitrogen monoxide may be carried out in such a way that, at a very high selectivity for cyclododecadienone, a conversion of cyclododecatriene in the range of up to 50%, preferably in the range from 5 to 30% and especially preferably in the range from 10 to 20% is achieved. The selectivity based on cyclododecadienone is generally at least 90%, preferably at least 92.5% and more preferably at least 95%.

In principle, it is possible to react any cyclododecatriene or any mixture of two and more different cyclododecatrienes with dinitrogen monoxide. Examples include 1,5,9-cyclododecatrienes, for example cis,trans,trans-1,5,9-cyclododecatriene or cis,cis,trans-1,5,9-cyclododecatriene or all-trans-1,5,9-cyclododecatriene.

The cyclododecatriene used is preferably cis,trans,trans-1,5,9-cyclododecatriene.

In general, the reaction of cis,trans,trans-1,5,9-cyclododecatriene with dinitrogen monoxide results in a cyclododeca-4,8-dienone isomer mixture which comprises at least two of the isomers cis,trans-cyclododeca-4,8-dienone, trans,cis-cyclododeca-4,8-dienone and trans,trans-cyclododeca-4,8-dienone. An example of a typical isomer mixture accordingly has the isomers in molar ratios of about 1:1:0.08.

The reaction of cyclododecatriene with dinitrogen monoxide can in principle be effected in the presence of a catalyst, but also without addition of a catalyst.

For the hydrogenation in stage (a-3), it is possible to use any suitable catalysts. In particular, it is possible to use at least one homogeneous or at least one heterogeneous catalyst or both at least one homogeneous and at least one heterogeneous catalyst.

The usable catalysts preferably comprise at least one metal from transition group 7, 8, 9, 10 or 11 of the Periodic Table of the Elements. Preference is further given to the catalysts usable in accordance with the invention comprising at least one element selected from the group consisting of Re, Fe, Ru, Co, Rh, Ir, Ni, Pd, Pt, Cu and Au. Special preference is given to the catalysts usable in accordance with the invention comprising at least one element selected from the group consisting of Fe, Ni, Pd, Pt and Cu. Particular preference is given to the catalysts usable in accordance with the invention comprising Pd, Pt, Ru or Ni.

Suitable catalysts are, for example, homogeneous catalysts comprising at least one element of transition group 8, 9 or 10. Preference is further given to homogeneous catalysts which comprise Ru, Rh, Ir and/or Ni. Examples which should be mentioned in this context are RhCl(TTP)₃ or Ru₄H₄(CO)₁₂. Particular preference is given to those homogeneous catalysts which comprise Ru. For example, homogeneous catalysts used are those described in U.S. Pat. No. 5,180,870, U.S. Pat. No. 5,321,176, U.S. Pat. No. 5,177,278, U.S. Pat. No. 3,804,914, U.S. Pat. No. 5,210,349, U.S. Pat. No. 5,128,296, U.S. Pat. No. 316,917 and in D. R. Fahey in J. Org. Chem. 38 (1973) p. 80-87, whose disclosure-content on this subject is incorporated fully into the context of the present application. Such catalysts are, for instance, (TPP)₂(CO)₃Ru, [Ru(CO)₄]₃, (TPP)₂Ru(CO)₂Cl₂, (TPP)₃(CO)RuH₂, (TPP)₂(CO)₂RuH₂, (TPP)₂(CO)₂RuClH or (TPP)₃(CO)RuCl₂.

Especially suitable is at least one heterogeneous catalyst, in which case it is possible to use at least one of the abovementioned metals in the form of the metal as such, in the form of Raney catalyst and/or applied to a customary support. Preferred support materials are, for instance, activated carbons or oxides, for example aluminas, silicas, titanias or zirconias. Other support materials which should be mentioned include bentonites. When two or more metals are used, they may be present separately or as an alloy. It is possible in this case to use at least one metal as such and at least one other metal in the form of Raney catalyst, or at least one metal as such and at least one other metal applied to at least one support, or at least one metal in the form of Raney catalyst and at least one other metal applied to at least one support, or at least one metal as such and at least one other metal in the form of Raney catalyst and at least one other metal applied to at least one support.

The catalysts used may, for example, also be what are known as precipitation catalysts. Such catalysts may be prepared by precipitating their catalytically active components from their salt solutions, in particular from the solutions of their nitrates and/or acetates, for example by adding solutions of alkali metal hydroxide and/or carbonate and/or alkaline earth metal hydroxide and/or carbonate solutions, for example sparingly soluble hydroxides, oxide hydrates, basic salts or carbonates, subsequently drying the resulting precipitates and then converting them by calcination at generally from 300 to 700° C., in particular from 400 to 600° C., to the corresponding oxides, mixed oxides and/or mixed-valence oxides, which are reduced by a treatment with hydrogen or with hydrogen-comprising gases in the range of generally from 50 to 700° C., in particular from 100 to 400° C., to the metals and/or low-oxidation state oxidic compounds in question and converted to the actual catalytically active form. Reduction is generally effected until no more water is formed. In the preparation of precipitation catalysts which comprise a support material, the catalytically active components can be precipitated in the presence of the support material in question. The catalytically active components may advantageously be precipitated from the salt solutions in question simultaneously with the support material.

Preference is given to using hydrogenation catalysts which comprise the metals or metal compounds catalyzing the hydrogenation deposited on a support material.

Apart from the abovementioned precipitation catalysts which, apart from the catalytically active components, also additionally comprise a support material, support materials suitable for the process according to the invention are generally those in which the catalytically hydrogenating component has been applied to a support material, for example, by impregnation.

The way in which the catalytically active metal is applied to the support is generally not critical and can be brought about in various different ways. The catalytically active metals may be applied to these support materials, for example, by impregnation with solutions or suspensions of the salts or oxides of the elements in question, drying and subsequent reduction of the metal compounds to the metals or low-oxidation state compounds in question by means of a reducing agent, preferably with hydrogen or complex hydrides. Another means of applying the catalytically active metals to these supports is to impregnate the supports with solutions of thermally readily decomposable salts, for example with nitrates or thermally readily decomposable complexes, for example carbonyl or hydrido complexes of the catalytically active metals, and to heat the thus impregnated support to temperatures in the range from 300 to 600° C. to thermally decompose the adsorbed metal compounds. This thermal decomposition is preferably undertaken under a protective gas atmosphere. Suitable protective gases are, for example, nitrogen, carbon dioxide, hydrogen or the noble gases. In addition, the catalytically active metals may be deposited on the catalyst support by vapor deposition or by flame spraying. The content in these supported catalysts of the catalytically active metals is in principle not critical for the success of the process according to the invention. In general, higher contents of catalytically active metals in these supported catalysts lead to higher space-time conversions than lower contents. In general, supported catalysts are used whose content of catalytically active metals is in the range from 0.1 to 90% by weight, preferably in the range from 0.5 to 40% by weight, based on the total weight of the catalyst. Since these content data are based on the overall catalyst including support material, but the different support materials have very different specific weights and specific surface areas, it is also conceivable for the contents to be lower or higher than these data, without this having a disadvantageous effect on the result of the process according to the invention. It will be appreciated that a plurality of the catalytically active metals may also be applied to the particular support material. In addition, the catalytically active metals may also be applied to the support, for example, by the process of DE-A 25 19 817, EP 1 477 219 A1 or EP 0 285 420 A1. In the catalysts according to the aforementioned documents, the catalytically active metals are present in the form of an alloy which is generated by thermal treatment and/or reduction of a salt or complex of the aforementioned metals which were applied for example, by impregnation.

Both the precipitation catalysts and the supported catalysts may also be activated in situ at the start of the reaction by the hydrogen present. Preference is given to separately activating these catalysts before their use.

The support materials used may generally be the oxides of aluminum and of titanium, zirconium dioxide, silicon dioxide, aluminas, for example montmorillonites, silicates, for example magnesium silicates or aluminum silicates, zeolites, for example of the ZSM-5 or ZSM-10 structure types, or activated carbon. Preferred support materials are aluminas, titanium dioxides, silicon dioxide, zirconium dioxide and activated carbon. It will be appreciated that mixtures of different support materials may also serve as the support for catalysts usable in the process according to the invention.

According to the invention, very particularly preferred catalysts are those which comprise Ni, Pt and/or Pd and are applied to a support. Very preferred supports are or comprise activated carbon, aluminum oxide, titanium dioxide and/or silicon dioxide.

The at least one heterogeneous catalyst may be used, for example, in the form of a suspension catalyst and/or in the form of a fixed bed catalyst.

When, for example, in the process according to the invention, the hydrogenation in stage (a-3) is carried out with at least one suspension catalyst, hydrogenation is effected preferably in at least one stirred reactor or in at least one bubble column or in at least one packed bubble column or in a combination of two or more identical or different reactors.

The term “different reactors” refers in the present context both to different reactor types and to reactors of the same type which differ, for example, by their geometry, for example their volume and/or their cross section, and/or by the hydrogenation conditions in the reactors.

When, for example, in the process according to the invention, the hydrogenation in (a-3) is carried out with at least one fixed bed catalyst, preference is given to using at least one tubular reactor, for example at least one shaft reactor and/or at least one tube bundle reactor, and it is possible to operate a single reactor in liquid phase mode or trickle mode. When two or more reactors are used, it is possible to operate at least one in liquid phase mode and at least one in trickle mode.

When the catalyst used in the hydrogenation is, for example, a heterogeneous catalyst as a suspension catalyst, preference is given in the context of the present invention to removing it by at least one filtration step. The catalyst removed in this way may be recycled into the hydrogenation or fed to at least one arbitrary other process. It is equally possible to work up the catalyst, in order, for example, to recover the metal present in the catalyst.

When the catalyst used in the hydrogenation in stage (a-3) is, for example, a homogeneous catalyst, preference is given to removing it in the context of the present invention by at least one distillation step. In this distillation, one or two or more distillation columns may be used. The catalyst removed in this way may be recycled into the hydrogenation or fed to at least one arbitrary other process. It is equally possible to work up the catalyst, in order, for example, to recover the metal present in the catalyst.

Before use in an arbitrary process, for example before recycling into the process according to the invention, both the at least one homogeneous and the at least one heterogeneous catalyst, can be regenerated, should this be necessary, by at least one suitable process.

The heat can be removed in the reactor used in accordance with the invention internally, for example via cooling coils, and/or externally, for example via at least one heat exchanger. When, for example and with preference, at least one tubular reactor is used for the hydrogenation, preference is given to conducting the reaction via external circulation, in which the heat removal is integrated.

When, in a preferred embodiment of the process according to the invention, the hydrogenation is carried out continuously, preference is further given to using at least two reactors, more preferably at least two tubular reactors, more preferably at least two serially coupled tubular reactors and especially preferably exactly two serially coupled tubular reactors. The hydrogenation conditions in each of the reactors used may be the same or different and are within the above-described ranges.

When the hydrogenation in stage (a-3) is carried out over at least one suspended catalyst, the residence time is generally in the range from 0.05 to 50 h, for example in the range from 0.5 to 50 h, preferably in the range from 1 to 30 h and more preferably in the range from 1.5 to 25 h, most preferably in the range from 1.5 to 10 h. It is unimportant whether, in accordance with the invention, one main reactor and one postreactor are used, or additionally further reactors. For all of these embodiments, the total residence time is within the above-specified ranges.

When, in the process according to the invention, the hydrogenation is carried out in continuous mode over at least one fixed bed catalyst, the catalyst hourly space velocity (kg of feed/liters of catalyst×h) is generally in the range from 0.03 to 20, preferably in the range from 0.05 to 5 and more preferably in the range from 0.1 to 2. It is unimportant whether, in accordance with the invention, one main reactor and one postreactor are used, or additionally further reactors. For all of these embodiments, the total hourly space velocity is within the above-specified ranges.

In general, the hydrogenation temperature in the main reactor is in the range from 0 to 350° C., preferably in the range from 20 to 300° C., more preferably in the range from 50 to 250° C. and especially preferably in the range from 80 to 220° C.

The hydrogen pressure in the inventive hydrogenation in the main reactor is generally in the range from 1 to 325 bar, preferably in the range from 5 to 300 bar, more preferably in the range from 10 to 250 bar and especially preferably in the range from 15 to 150 bar.

In the inventive hydrogenation in stage (a-3), at least one suitable solvent or diluent may be used. In principle, these include all solvents and diluents which are not hydrogenated or converted in another way under the hydrogenation conditions, for example alcohols, ethers, hydrocarbons, water, aromatics or ketones, in particular toluene or cyclododecane.

In a preferred embodiment of the process according to the invention, the hydrogenation in stage (a-3) is carried out without addition of a solvent or diluent.

In a further embodiment, the composition (I) comprising cyclododecanone can be obtained by trimerizing butadiene to cyclododecatriene, hydrogenating cyclododecatriene to cyclododecane and subsequently oxidizing cyclododecane to cyclododecanone.

In a further embodiment, the present invention therefore relates to a process as described above for purifying a composition (I) at least comprising cyclododecanone, wherein the composition (I) is obtainable by a process at least comprising the stages of
(a-I) trimerization of butadiene to cyclododecatriene
(a-II) hydrogenation of cyclododecatriene to cyclododecane or cyclododecene
(a-II) oxidation of cyclododecane or cyclododecene to cyclododecanone.

According to the invention, further treatments, for example purification steps, may be effected between stages (a-I), (a-II) and (a-III). With regard to stage (a-I), the remarks made above for stage (a-I) apply. With regard to stage (a-II) and stage (a-III), reference is made to DE 103 44 594 A, whose contents on this subject are incorporated fully into the context of the present application.

It is equally possible in the context of the present invention that the composition (I) comprising cyclododecanone is obtained by trimerizing butadiene to cyclododecatriene, oxidizing cyclododecatriene to cyclododecatriene epoxide and subsequently hydrogenating and rearranging cyclododecatriene epoxide to cyclododecanone.

In a further embodiment, the present invention therefore relates to a process as described above for purifying a composition (I) at least comprising cyclododecanone, wherein the composition (I) is obtainable by a process at least comprising the stages of
(a-A) trimerization of butadiene to cyclododecatriene
(a-B) oxidation of cyclododecatriene to cyclododecatriene epoxide
(a-C) hydrogenation of cyclododecatriene epoxide and rearrangement to cyclododecanone.

According to the invention, further treatments, for example purification steps, may be effected between stages (a-A), (a-B) and (a-C). With regard to stage (a-A), the remarks made above for stage (a-1) apply. With regard to stage (a-B) and stage (a-C), reference is made to EP 1 018 498 A2, whose contents on this subject are incorporated fully into the context of the present application.

The invention will be illustrated in detail below by examples.

### EXAMPLES

### Example 1

Crude cyclododecanone having a purity of about 98.5% cyclododecanone, prepared by a process according to DE 103 44 595 A, is distilled at 10 mbar. This affords a pure product (99.8% cyclododecanone) having a color number of 40 APHA. The product is irradiated with a 100 watt incandescent lamp at 65° C. for 1 hour. Subsequently, the color number is 10 APHA.

1-9. (canceled)

10. A process for purifying a composition (I) at least comprising cyclododecanone, at least comprising step (i)
(i) irradiation of the composition (I).

11. The process according to claim 10, wherein the irradiation is effected with light of a wavelength of from 200 to 800 nm.

12. The process according to claim 10, wherein the irradiation is effected for from 0.1 to 48 hours.

13. The process according to claim 10, wherein the composition (I) comprises at least one cyclic diketone.

14. The process according to claim 10, wherein the composition (I), after the irradiation according to step (i), has a color number of from 0 to 40 APHA.

15. The process according to claim 10, which comprises at least one step (ii):
(ii) distillation and/or crystallization of the composition (I).

16. The process according to claim 10, wherein the composition (I) is obtainable by a process at least comprising the stages of
(a-1) trimerization of butadiene to cyclododecatriene
(a-2) oxidation of cyclododecatriene to cyclododecadienone
(a-3) hydrogenation of cyclododecadienone to cyclododecanone.

17. The process according to claim 10, wherein the composition (1) is obtainable by a process at least comprising the stages of
(a-I)trimerization of butadiene to cyclododecatriene
(a-II) hydrogenation of cyclododecatriene to cyclododecane or cyclododecene
(a-III) oxidation of cyclododecane or cyclododecene to cyclododecanone.

18. The process according to claim 10, wherein the composition (I) is obtainable by a process at least comprising the stages of
(a-A) trimerization of butadiene to cyclododecatriene
(a-B) oxidation of cyclododecatriene to cyclododecatriene epoxide
(a-C) hydrogenation of cyclododecatriene epoxide and rearrangement to cyclododecanone.

19. A process for purifying a composition (1) at least comprising cyclododecanone, at least comprising step (i)
(i) irradiation of the composition (1);
wherein the composition (I) comprises at least one cyclic diketone.

20. The process according to claim 19, which comprises at least one step (ii):
(ii) distillation and/or crystallization of the composition (1).

21. The process according to claim 19, wherein the composition (1) is obtainable by a process at least comprising the stages of
(a-1) trimerization of butadiene to cyclododecatriene
(a-2) oxidation of cyclododecatriene to cyclododecadienone
(a-3) hydrogenation of cyclododecadienone to cyclododecanone.

22. The process according to claim 19, wherein the composition (1) is obtainable by a process at least comprising the stages of
(a-I) trimerization of butadiene to cyclododecatriene
(a-I) hydrogenation of cyclododecatriene to cyclododecane or cyclododecene
(a-III) oxidation of cyclododecane or cyclododecene to cyclododecanone.

23. The process according to claim 19, wherein the composition (1) is obtainable by a process at least comprising the stages of
(a-A) trimerization of butadiene to cyclododecatriene
(a-B) oxidation of cyclododecatriene to cyclododecatriene epoxide
(a-C) hydrogenation of cyclododecatriene epoxide and rearrangement to cyclododecanone.

24. A process for purifying a composition (I) at least comprising cyclododecanone, at least comprising step (i)
(i) irradiation of the composition (I);
which comprises at least one step (ii):
(ii) distillation and/or crystallization of the composition (1).

25. The process according to claim 11, wherein the irradiation is effected for from 0.1 to 48 hours.

26. The process according to claim 11, wherein the composition (1) comprises at least one cyclic diketone.

27. The process according to claim 12, wherein the composition (1) comprises at least one cyclic diketone.

28. The process according to claim 11, wherein the composition (1), after the irradiation according to step (i), has a color number of from 0 to 40 APHA.

29. The process according to claim 12, wherein the composition (1), after the irradiation according to step (i), has a color number of from 0 to 40 APHA.
